SEQUENCE LISTING (51 sequences)

SEQ ID NO: 1 - protein, 10 aa, Homo Sapiens
GDSVSSNTAA

SEQ ID NO: 2 - protein, 9 aa, Homo Sapiens
TYHRSKWVT

SEQ ID NO: 3 - protein, 11 aa, Homo Sapiens
ARDIGLADFDY

SEQ ID NO: 4 - protein, 9 aa, Homo Sapiens
SSSNIESNY

SEQ ID NO: 5 - protein, 3 aa, Homo Sapiens
RNY

SEQ ID NO: 6 - protein, 11 aa, Homo Sapiens
GAWDDGPSGWV

SEQ ID NO: 7 - protein, 217 aa, Homo Sapiens
QSGPGLVRPSQTLSLTCVISGDSVSSNTAAWNWIRQSPSRGLEWLGRTYHRSKWVTDYAAFVRSRIAINSDTSKNQFSLRLNSVTPEDTAVYYCARDIGLADFDYWGQGTLVTVSPASPTSPKVFPLSLDSTPQDGNVVVACLVQGFFPQEPLSVTWSESGQGVTARNFPPSQDASGDLYTTSSQLTLPATQCLAGKSVTCHVKHYTNPSQDVTVPA

SEQ ID NO: 8 - protein, 216 aa, Homo Sapiens
QSVLTQPPSASGTPGQRVTISCSGSSSNIESNYVSWYQHLPGSAPKLLIYRNYQRPSGVPDRFSGSKSGTSASLAISGLRSEDEADYYCGAWDDGPSGWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 9 - DNA, 654 bp, Homo Sapiens
cagtcaggtccaggactggtgaggccctcgcagaccctctcactcacctgtgtcatctccggggacagtgtctctagcaacactgctgcttggaactggatcaggcagtccccatcgagaggccttgagtggctgggaaggacataccacaggtccaagtgggttactgattatgcagcgtttgtgagaagtcgaatagccatcaactcagacacatccaagaaccaattctccctgcggctgaactctgtgactcccgaggacacggctgtgtattactgtgccagagatatagggctggcggactttgactactggggccagggaaccctggtcaccgtctccccggccccgaccagccccaaggtcttcccgctgagcctcgacagcaccccccaagatgggaacgtggtcgtcgcatgcctggtccagggcttcttcccccaggagccactcagtgtgacctggagcgaaagcggacagggcgtgaccgccagaaacttcccacccagccaggatgcctccggggacctgtacaccacgagcagccagctgaccctgccggccacacagtgcctagccggcaagtccgtgacatgccacgtgaagcactacacgaatcccagccaggatgtgactgtgcctgcctaataa

SEQ ID NO: 10 - DNA, 651 bp, Homo Sapiens
cagtctgtgctgactcagccaccctcagcgtctgggacccccgggcagagggtcaccatctcttgttctggaagcagctccaacatcgaaagtaattatgtatcttggtatcagcacctcccaggatcggcccccaaactcctcatctataggaattatcagcggccctcaggggtccctgaccgattctctggctccaagtctggcacctcagcctccctggccatcagtgggctccggtccgaggatgaggctgattattactgtggagcatgggatgacggcccgagtggttgggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttcataa

SEQ ID NO: 11 - protein, 144 aa, Human Immunodeficiency Virus Type 1
QARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIK

SEQ ID NO: 12 - protein, 35 aa, Human Immunodeficiency Virus Type 1
SQTQQEKNEQELLELDKWASLWNWFDITNWLWYIK

SEQ ID NO: 13 - protein, 122 aa, Artificial Sequence
  Polypeptide Gp41-engineered loop
MQARQLLSGIVQQQNNLLRAIEAQQHLLQLTVWGIKQLQAAILAVERYLKDQQLLGIWGSSGGRGGSNASWSNKSLEQIWNHTTWMEWREINNYTSLIHSLIEESQNQQEKNEQELLELDLE

SEQ ID NO: 14 - DNA, 24 bp, Artificial Sequence
  Primer
caggtgcagctcgagcagtctggg

SEQ ID NO: 15 - DNA, 24 bp, Artificial Sequence
  Primer
caggtgcagctgctcgagtctggg

SEQ ID NO: 16 - DNA, 24 bp, Artificial Sequence
  Primer
caggtcaccttgctcgagtctggt

SEQ ID NO: 17 - DNA, 24 bp, Artificial Sequence
  Primer
gaggtgcagctcgaggagtctggg

SEQ ID NO: 18 - DNA, 24 bp, Artificial Sequence
  Primer
gaggtgcagctgctcgagtctggg

SEQ ID NO: 19 - DNA, 23 bp, Artificial Sequence
  Primer
caggtgcagctgctcgagtcggg

SEQ ID NO: 20 - DNA, 23 bp, Artificial Sequence
  Primer
caggtgcagctactcgagtgggg

SEQ ID NO: 21 - DNA, 23 bp, Artificial Sequence
  Primer
gaggtgcagctcgagcagtctgg

SEQ ID NO: 22 - DNA, 23 bp, Artificial Sequence
  Primer
caggtacagctcgagcagtcagg

SEQ ID NO: 23 - DNA, 23 bp, Artificial Sequence
  Primer
caggtgcagctcgagcaatctgg

SEQ ID NO: 24 - DNA, 30 bp, Artificial Sequence
  Primer
agttgaactagttgggcagggcacagtcac

SEQ ID NO: 25 - DNA, 30 bp, Artificial Sequence
  Primer
agttgaactagttcggcagggaacagtcac

SEQ ID NO: 26 - DNA, 24 bp, Artificial Sequence
  Primer
gacatcgagctcacccagtctcca

SEQ ID NO: 27 - DNA, 23 bp, Artificial Sequence
  Primer
gacatcgagctcacccagtctcc

SEQ ID NO: 28 - DNA, 24 bp, Artificial Sequence
  Primer
gatattgagctcactcagtctcca

SEQ ID NO: 29 - DNA, 18 bp, Artificial Sequence
  Primer
gatattacccagactcca

SEQ ID NO: 30 - DNA, 24 bp, Artificial Sequence
  Primer
gaaattgagctcacgcagtctcca

SEQ ID NO: 31 - DNA, 24 bp, Artificial Sequence
  Primer
gaaattgagctcacncagtctcca

SEQ ID NO: 32 - DNA, 18 bp, Artificial Sequence
  Primer
gacatcacccagtctcca

SEQ ID NO: 33 - DNA, 24 bp, Artificial Sequence
  Primer
gaaacggagctcacgcagtctcca

SEQ ID NO: 34 - DNA, 24 bp, Artificial Sequence
  Primer
gaaattgagctcactcagtctcca

SEQ ID NO: 35 - DNA, 26 bp, Artificial Sequence
  Primer
cagtctgagctcacgcagccnccctc

SEQ ID NO: 36 - DNA, 26 bp, Artificial Sequence
  Primer
cagtctgagctcactcagcctgcctc

SEQ ID NO: 37 - DNA, 24 bp, Artificial Sequence
  Primer
gcctcctatgagctcactcagcca

SEQ ID NO: 38 - DNA, 26 bp, Artificial Sequence
  Primer
cagcctgagctcactcaatcatcctc

SEQ ID NO: 39 - DNA, 26 bp, Artificial Sequence
  Primer
cagcctgagctcactcagcccccgtc

SEQ ID NO: 40 - DNA, 27 bp, Artificial Sequence
  Primer
cagcctgagctcactcagccgncttcc

SEQ ID NO: 41 - DNA, 24 bp, Artificial Sequence
  Primer
aattttgagctcactcagccccac

SEQ ID NO: 42 - DNA, 24 bp, Artificial Sequence
  Primer
cagactgagctcactcaggagccc

SEQ ID NO: 43 - DNA, 24 bp, Artificial Sequence
  Primer
cagactacccaggagccatcgttc

SEQ ID NO: 44 - DNA, 26 bp, Artificial Sequence
  Primer
cagcctgagctcactcagccaccttc

SEQ ID NO: 45 - DNA, 27 bp, Artificial Sequence
  Primer
caggcagagctcactcagccaccctcg

SEQ ID NO: 46 - DNA, 36 bp, Artificial Sequence
  Primer
tccttctagattactaacactctcccctgttgaagc

SEQ ID NO: 47 - DNA, 30 bp, Artificial Sequence
  Primer
cgccgtctagaattatgaacattctgtagg

SEQ ID NO: 48 - DNA, 22 bp, Artificial Sequence
  Primer
aagacagctatcgcgattgcag

SEQ ID NO: 49 - DNA, 21 bp, Artificial Sequence
  Primer
acctattgcctacggcagccg

SEQ ID NO: 50 - DNA, 22 bp, Artificial Sequence
  Primer
gagcagctgcacctcggccatg

SEQ ID NO: 51 - DNA, 24 bp, Artificial Sequence
  Primer
gcccccttattagcgtttgccatc

(19) United States
(12) Patent Application Publication
(10) Pub. No.: US 2009/0232826 A1
(43) Pub. Date: Sep. 17, 2009

(54) Antibody or a fragment thereof, having neutralizing activity against hiv but not against il2

(75) Inventors: Morgane Bomsel, Paris (FR); Daniela Tudor, Fontenay Aux Roses (FR)

Correspondence Address: OLIFF & BERRIDGE, PLC, P.O. BOX 320850, ALEXANDRIA, VA, 22320-4850, US

(73) Assignees: MYMETICS CORPORATION, NEW YORK, NY (US); INSTITUT NATIONAL DE LA SANTE ET DE LA RECHERCHE MEDICALE (INSERM), PARIS (FR)

(21) Appl. No.: 11/919,698

(22) PCT Filed: May 2, 2005
(86) PCT No.: PCT/IB2005/001182

§ 371 Date: Jul. 18, 2008

(51) Int. Cl. A61K 39/395; C07K 16/00; C07K 16/08; C07H 21/00; C12N 15/63; C12N 5/10

(52) U.S. Cl. 424/141.1; 530/388.1; 530/387.9; 530/387.3; 536/23.53; 435/320.1; 435/325

(57) ABSTRACT

The invention relates to a monoclonal antibody or a fragment thereof comprising in the H chain variable region at least one complementarity determining region (CDR) selected among CDR1, CDR2 and CDR3 having, respectively, an amino acids sequence set forth as SEQ ID NO 1, SEQ ID NO 2, SEQ ID NO 3 or functional analogues thereof.

The instant invention relates to an antibody or a fragment thereof, having neutralizing activity against HIV, and useful in the treatment and/or prevention of HIV infection. The invention also relates to a pharmaceutical composition comprising said antibody, a diagnostic composition and a method for providing passive immunotherapy.

Human immunodeficiency virus (HIV) is a member of the lentivirus family of animal retroviruses and is a causative agent of Acquired Immune Deficiency Syndrome (AIDS). To date, two closely related types of HIV type 1 (HIV-1) and type 2 (HIV-2) have been identified and characterized at the molecular level.

The introduction of antiviral agents against HIV, such as reverse transcriptase inhibitors, has allowed to greatly improve the conditions of the patient affected by AIDS. However, in most cases, therapeutic efficacy of these drugs to AIDS is partial or temporal, and in addition, these drugs exhibit toxicity or growth inhibition to haematopoietic cells, and thereby inhibit reconstruction of an immune system which has become deficient.

Therefore, it is generally agreed that AIDS prevention programs and antiretroviral drug therapies (VALDISERRI, 2003, Nat. Med, 9:881) should be combined with effective microbicides and vaccines. But the design and testing of such vaccines have proven to be complex (LETVIN, et al. 2002, Annu. Rev. Immunol., 20:73; McMICHAEL & HANKE, 2003, Nat. Med., 9:874).

Mucosal surfaces are the major site for HIV-1 entry (NICOLOSI, et al. 1994, J. Acquir. Immun. Defic. Syndr., 7:296). HIV-1 transmission may occur through exposure of mucosal surfaces to HIV-1 infected fluids, such as semen, colostrums, breast milk, and cervico-vaginal fluid (CHERMANN, 1998, Am. J. Reprod. Immunol., 40:183; MILMAN & SCHARMA, 1994, AIDS, 10:1305).

Because of the interaction of HIV with mucosal surface, a component of a vaccine designed against HIV should engage the mucosal immune system to interfere with early steps of mucosal viral transmission and potential receptors.

It has been recognized that antibodies neutralizing AIDS viruses may clearly play an important role in protection.

However, while it was possible to achieve neutralizing antibodies against a specific virus strain grown in the laboratory, the carrying out of antibodies that could neutralized a broad array of strains and that could be effectively active in vivo has not yet met similar success.

The ectodomain of gp41 of HIV being the most conserved region in the viral envelope is a extremely immunogenic glycoprotein.

It is known from U.S. Pat. No. 6,455,265 that conserved and immunodominant regions of the retroviral envelope protein gp41 of the HIV virus could be responsible for harmful autoimmune phenomena, due to three-dimensional structural analogies and/or cross-reactivity with certain regions of a protein of the human immune system, and in particular IL-2.

From WO 2005/010033, are known gp41 engineered loop proteins comprising in the connecting loop between N- and C-helices of gp41 a linker fragment and being devoid of or having reduced autoimmune side effects.

Therefore, there is a need for an antibody that allows for neutralizing HIV infection, and in particular HIV-1 infection, without autoimmune side effect.

There is a need for an antibody that allows to prevent and/or reduce the HIV infection through mucosal surface.

There is also a need for providing an antibody for the manufacture of a medicament intended to be used in passive immunotherapy.

There is a need for an antibody that may be used for diagnostic purposes.

The invention has for object to satisfy to all or part of those above-mentioned needs.

The inventors have obtained an efficient monoclonal antibody that allows to satisfy the above-mentioned unmet needs.

In particular, the inventors have identified a novel IgA antibody Fab comprising in the H chain variable region at least one complementary determining region (CDR) selected among CDR1, CDR2 and CDR3 having respectively an amino acid sequence set forth as SEQ ID NO:1, SEQ ID NO:2 and SEQ ID NO:3 or functional analogues thereof.

More particularly, the novel identified IgA antibody Fab may recognize a gp41 engineered loop protein or a wild-type gp41 protein but may not recognize a peptide called P1 which occurs in the gp41 engineered loop protein and the wild-type gp41 protein.

The peptide P1 (SEQ ID NO:12) corresponds, for example, to the amino acid sequence 649 to 683 of the wild-type amino acid sequence of the gp41 protein set forth as SEQ ID NO:11, and obtained from HIV-1 strain HxB2.

The newly identified antibody has also the ability to inhibit HIV-1 transcytosis and block the CD4+ T cells infection.

Therefore according to one of its aspects, the instant invention is related to a monoclonal antibody or a fragment thereof comprising in the H chain variable region at least one complementary determining region (CDR) selected among CDR1, CDR2 and CDR3 having, respectively, an amino acid sequence set forth as SEQ ID NO:1, SEQ ID NO:2 and SEQ ID NO:3 or functional analogues thereof.

According to another of its aspects, the instant invention is related to a monoclonal antibody or a fragment thereof, recognizing a gp41 engineered loop protein as defined thereafter and not recognizing a peptide of the amino acid sequence set forth as SEQ ID NO:12. As example of gp41 engineered loop protein liable to be recognized by an antibody according to the invention mention may be made of the gp41 engineered loop protein having the amino acid sequence set forth as SEQ ID NO:13.

Accordingly, an antibody of the invention may also recognize a wild-type sequence of gp41 protein which may naturally occur in various strains of HIV.

According to another of its aspects, the instant invention is related to a monoclonal antibody or a fragment thereof comprising a L chain variable region comprising at least one complementary determining region (CDR) selected among CDR1, CDR2 and CDR3 having respectively an amino acid sequence set forth as SEQ ID NO:1, SEQ ID NO:2 and SEQ ID NO:3 or functional analogues thereof.

The terms “antibody fragment” as used herein refers to an antibody that has an amino-terminal and/or carboxy-terminal deletion but where the remaining amino acid sequence is identical to the corresponding positions in the naturally occurring sequence and which has its biological properties maintained or non adversely affected. This antibody fragment may comprise additional modifications such as insertion, deletion and/or substitution of amino acids residues and/or fusion with other peptides or proteins to make chimeric proteins. The term “antibody fragment” may also encompass the various parts of an antibody, i.e. the constant, variable, heavy and light chains.

Within the meaning of the invention, the expression “functional analogue” with respect to a peptide is intended to refer to a peptide that has an amino acid sequence homology or identity with another amino acid sequence and that has similar or conserved biological properties relative to said other peptide sequence. Typically, peptide analogues comprised conservative amino acid substitutions (and/or insertions and/or deletions) with respect to the naturally-occurring sequence.

According to another aspect, the instant invention also relates to an H chain variable region comprising at least one CDR selected among CDR1, CDR2 and CDR3 having respectively an amino acid sequence set forth as SEQ ID NO:3, SEQ ID NO:4, and SEQ ID NO:5 or functional analogues thereof.

According to another of its aspects, the instant invention is directed to an H chain variable region recognizing a gp41 engineered loop protein as defined thereafter, and for example having the peptide sequence set forth as SEQ ID NO:13, and not recognizing a peptide of the amino acid sequence set forth as SEQ ID NO:12.

According to another of its aspects, the antibodies of the invention or fragments thereof have the ability to neutralize HIV.

According to another of its aspects, the instant invention also concerns nucleic acids sequence encoding for the antibody of the invention or a fragment thereof, as well as an expression vector and host cell comprising said nucleic acids sequence.

According to another of its aspects, the instant invention relates to a pharmaceutical composition comprising as active agent an effective amount of an agent selected among an antibody of the invention or a fragment thereof, a nucleic acids sequence of the invention, a vector of the invention or a host cell of the invention, and a suitable carrier.

According to another of its aspects, the instant invention is also directed to a diagnostic composition and a method for detecting, in vitro, a HIV strain in a sample.

According to another of its aspects, the instant invention also concerns a method for providing passive immunotherapy to an individual liable to be infected with HIV comprising administering a therapeutically effective amount of at least an antibody of the invention or a fragment thereof.

### Antibodies

An antibody according to the invention refers to an intact immunoglobulin or a fragment thereof, and in particular to an antigen binding portion thereof.

An immunoglobulin is a tetrameric molecule, composed of two identical pairs of polypeptide chains, each pair having one “light” (L) (about 25 kDa) and one “heavy” (H) chain (about 50-70 kDa). The amino-terminal portion of each chain includes a variable region (V) of about 100 to 110 or more amino acids, primarily responsible for antigen recognition. The carboxy-terminal portion of each chain defines a constant region (C) primarily responsible for effector function. Human light chains are classified as κ and λ light chains. Heavy chain constant regions are classified as μ, δ, γ, α, or ε, and define the antibodies isotype as IgM, IgD, IgG, IgA and IgE respectively. The variable regions of each light/heavy chain pair form the antibody binding site, such that an intact immunoglobulin generally has at least two binding site.

In a particular embodiment of the invention, a monoclonal antibody of the invention or fragment thereof, may be an IgA, and more particularly a secretary IgA (S-IgA).

According to another embodiment, a monoclonal antibody of the invention or a fragment thereof may be a human antibody.

Immunoglobulin chains display the same general structure of relatively conserved framework regions (FR) joint by free hypervariable regions also called complementarity determining regions or CDRs. The CDRs from the two chains of each pair are aligned by the framework regions, enabling binding to a specific epitope (antigen-binding portion of the antibody). From N-terminus to C-terminus, both light and heavy chains comprise the domains FR1, CDR1, FR2, CDR2, FR3, CDR3 and FR4.

According to an embodiment, an antibody according to the invention may comprise in the H chain variable region at least one complementarity determining region (CDR) selected among CDR1, CDR2 and CDR3 having, respectively, an amino acid sequence set forth as SEQ ID NO:1, SEQ ID NO:2 and SEQ ID NO:3 or functional analogues thereof.

According to a particular embodiment, the H chain variable region of an antibody of the invention or a fragment thereof may comprise as CDRs, the CDR1, CDR2 and CDR3 having, respectively, an amino acid sequence set forth as SEQ ID NO:1, SEQ ID NO:2 and SEQ ID NO:3, or functional analogues thereof.

Within another embodiment, an antibody of the invention is also an H chain variable region as previously defined. In particular, an H chain variable region of the invention may comprise at least one CDR selected among CDR1, CDR2 and CDR3 having respectively an amino acid sequence set forth as SEQ ID NO:1, SEQ ID NO:2, and SEQ ID NO:3 or functional analogues thereof.

According to another embodiment, an H chain variable region of the invention may comprise as CDRs, the CDR1, CDR2 and CDR3 having, respectively, an amino acid sequence set forth as SEQ ID NO:1, SEQ ID NO:2, and SEQ ID NO:3, or functional analogues thereof.

An antibody according to the invention, or an H chain variable region of the invention may not recognize a peptide called P1 and having the amino acid sequence set forth as SEQ ID NO:12.

Within another embodiment of the instant invention, an antibody according to the invention or a fragment thereof, may also comprise a L chain variable region comprising at least one complementarity determining region selected among CDR1, CDR2 and CDR3 having, respectively, an amino acid sequence set forth as SEQ ID NO:4, SEQ ID NO:5, and SEQ ID NO:6 or functional analogues thereof.

Therefore, according to another embodiment, the instant invention also relates to a L chain variable region comprising at least one CDR selected among CDR1, CDR2 and CDR3, having respectively an amino acid sequence set forth as SEQ ID NO:4, SEQ ID NO:5, and SEQ ID NO:6 or functional analogues thereof.

According to another embodiment, a L chain variable region of the invention comprises as CDRs the CDR1, CDR2 and CDR3 having, respectively, an amino acid sequence set forth as SEQ ID NO:4, SEQ ID NO:5, and SEQ ID NO:6, or functional analogues thereof.

According to an embodiment, an antibody according to the invention comprises the H chain variable region and the L chain variable region having respectively the amino acid sequence set forth as SEQ ID NO:7 and SEQ ID NO:8 or functional analogues thereof. In a particular embodiment, an antibody of the invention is a Fab identified as clone 69 in the experimental section, and comprising said H and L chains variable regions.

According to another embodiment, an antibody of the invention may be a recombinant anti-HIV antibody or a fragment thereof. The recombinant antibody may comprise an H chain variable region of the invention.

According to another embodiment, a recombinant antibody of the invention may comprise, additionally, the L chain variable region of the invention.

Hence, the CDRs of the H chain variable region of the invention, or the H chain variable region as a whole, may be used to construct a chimeric or a recombinant antibody having a framework distinct of the IgA isotype, such as for example a framework of IgG isotype. Such construction may be obtained by any molecular biology tools known in the art, such as described in “Molecular Cloning—A Laboratory Manual” (2^{nd} ed.), Sambrook et al., 1989, Coldspring Harbor Laboratory, Coldspring Harbor Press, N.Y. (Sambrook). A chimeric or recombinant antibody may be a whole antibody or a fragment thereof.

Accordingly, a functional analogue of a CDR of the H chain variable region or of the L chain variable region of the invention, once inserted in lieu of the original sequence in an antibody according to the invention, maintains the ability of said antibody, or does not adversely affect its ability, to recognize a gp41 engineered loop protein as defined thereafter, or a wild-type gp41 protein. As example, the gp41 engineered loop protein may have an amino acid sequence set forth as SEQ ID NO:13. and the wild-type sequence of gp41 protein may have, for example, an amino acid sequence set forth as SEQ ID NO:11, or functional analogues thereof. Additionally, its ability to not recognize the peptide of sequence set forth as SEQ ID NO:12, and its ability to neutralize HIV and/or to inhibit a HIV infection are as well maintained.

The present invention also relates to antibodies comprising a Fab fragment derived from a human antibody of this invention and the human Fc domain derived from another Ig subtype, such as IgG and the like.

Therefore, according to an embodiment, the invention is also directed to an antigen binding portion of an antibody according to the invention or a fragment thereof, comprising a H chain variable region as previously defined. This antigen binding portion may optionally comprise a L chain variable region as previously defined.

Antigen-binding portions may be produced by any known recombinant DNA techniques or by enzymatic or chemical cleavage of intact antibodies. Antigen-binding portions may include, inter alia, Fab (monovalent fragment consisting of the VL, VH, CL and CH1 domains), F(ab′)₂ (bivalent fragment comprising two Fab fragments linked by a disulfide bridge at the hinge region), Fv (fragment consisting of the VL and VH domains of a single arm of an antibody), Fd (fragment consisting of the VH and CH1 domains), dAb fragment (consisting of a VH domain), scFv (consisting of an antibody in which VL and VH regions are paired to form a monovalent molecules via a synthetic linker), chimeric antibody, diabodies, and complementarity determining region (CDR) fragments.

The ability of the antibody of the invention or a fragment thereof, to neutralize HIV may be evaluated through the inhibition of transcytosis assay and the blocking of CD4+ T cells infection assay as described below and illustrated by the examples.

### GP41 Engineered Loop Protein

An antibody of the invention may recognize gp41 engineered loop proteins derived from the wild-type amino acid sequences of the gp41 glycoproteins of HIV. As example of wild-type amino acid sequence of gp41 protein that may convene to implement the instant invention, mention may be made of the amino acid sequence set forth as SEQ ID NO:11, obtained from the HIV-1 strain HxB2.

The gp41 engineered loop proteins that may be useful to carry out the instant invention may be obtained by introducing in the immunodominant regions some mutations (deletion, substitution and/or insertion) in order to reduce the homology with the human interleukine-2 (IL-2) as to avoid or reduce the risk of triggering an autoimmune reaction. Such gp41 engineered loop proteins are in particular described in WO 2005/010033 which is incorporated herein by reference.

In the present application, “mutation” refers to any modification of a region (optionally reduced to a single amino acid residue) of a polypeptide, by physical means, chemical means (covalent or noncovalent modification) and/or biological means (mutations by substitution, deletion and/or insertion of one or more amino acids), leading to the modification of the functional potentials of the constituent amino acid(s) of said region, termed “mutated region”. By way of example, it is possible to carry out mutations leading to the abolition, acquisition and/or modulation of the properties of disulfide bridges, hydrogen bonds, electrostatic interactions and/or hydrophobic interactions, the modification of the capacity of a protein to form a heterocomplex, or alternatively, in the case of an oligomeric protein, the modification of the state of oligomerization or of the stability of the oligomer.

The modification of the immunodominant regions results in the introduction in, or replacement of part of, the loop with a hydrophilic and non or weakly immunogenic, flexible linker and optionally with the introduction of mutation(s).

The gp41 engineered loop protein may include furthermore at least one mutation in its immunodominant region, which give in vitro a cross-reaction, of the B type and/or the T type, with a host protein and in particular with IL-2.

Some of the mutations decisive to impact this change in the antigenicity are disclosed in U.S. Pat. No. 6,455,265 and WO 2005/010033 which teachings are incorporated by reference herein in their entirety.

The gp41 engineered loop proteins useful for the invention may also include modifications such as truncation of a part of the amino acid sequence at the N- or C-terminal extremities or addition of peptide sequence to produce chimeric protein, such as a His-Tag or a HA-Tag, as described in WO 2005/010033.

To prepare the gp41 engineered loop proteins to be used in the instant invention, it is possible to use any known methods of peptide synthesis or genetic engineering techniques, such as described in “Molecular Cloning—A Laboratory Manual” (2^{nd} ed.), Sambrook et al., 1989, Coldspring Harbor Laboratory, Coldspring Harbor Press, N.Y. (Sambrook).

According to a particular embodiment, antibodies of the invention may recognize the gp41 engineered loop protein having an amino acid sequence set forth as SEQ ID NO:13, or analogues thereof.

Additionally, an antibody according to the invention or a fragment thereof, not only recognize a gp41 engineered loop protein having, for example, an amino acid sequence set forth as SEQ ID NO:13, but also recognizes the wild-type gp41 proteins, still without recognizing the peptide of sequence set forth as SEQ ID NO:12.

As example of a wild-type gp41 protein which may be recognized by an antibody of the invention mention may be made of the gp41 protein having the amino acid sequence set forth as SEQ ID NO:11, which is derived from the HIV strain HxB2 and which corresponds to the amino acid sequence from 540 to 683.

### The Peptide P1

The antibodies of the invention does not recognize a peptide called P1 and having the peptide sequence set forth as SEQ ID NO:12, or an analogue thereof.

The peptide P1 corresponds to an amino acid sequence present in the HIV envelope protein gp41 that is exposed at the surface of the viral particles before the viruses interact with target cells. As example, in the HIV-1 HxB2 strain, this sequence is comprised from amino acid 649 to amino acid 683 of the wild-type gp41 protein.

In aqueous solution, this peptide may adopt a structured, concentration dependent oligomeric state, namely dimeric or tetrameric state (ALFSEN & BOMSEL, 2002, J. Biol. Chem., 277: 25649).

Therefore, an antibody or an H chain variable region according to the invention or a fragment thereof does not recognize the peptide P1 under a monomeric or an oligomeric form.

### Neutralizing Activity of the Antibody

The neutralizing activity of the antibody of the invention, or fragment thereof, against HIV may be evaluated by the inhibition of HIV transcytosis across epithelial cells and/or inhibition of HIV infection of CD4+ T cells.

According to an embodiment, the neutralized HIV is more particularly a HIV-1 strain.

The evaluation of ability of the monoclonal antibody of the invention to inhibit the transcytosis of HIV across cells may be performed on any polarized cells. In one embodiment, the polarized cells may be epithelial cells, such as for example the intestinal cell line HT-29 or the endometrial cell line HEC-1 or cells from a human mucosal biopsy (BOMSEL et al., Immunity, 1998, 3:277). Typically, the cell lines are grown as a tight polarized monolayer on a permeable filter support (having for example 0.45 μm pore size) forming the interface between two independent chambers, the upper one bathing the apical surface of the epithelial monolayer and the lower one bathing the basolateral surface or the biopsies are mounted in using chambers.

Transcytosis may be initiated by contacting HIV-infected cells, as for example HIV-1-infected peripheral blood mononuclear cells (PBMC), at the apical chamber.

The antibody to be tested may be applied at the apical chamber before or after the application of the HIV infected cells or may be preincubated with the virus HIV-1 or HIV-1 infected cells.

The antibody of the invention to be tested may be applied at various concentrations, for example from about 0.01 to about 10 ng/ml, in particular from about 0.1 to about 5 ng/ml or more preferably at about 0.5 to about 1 ng/ml.

The evaluation of the virus transcytosis, and possibly its inhibition, may be carried out by the detection of HIV nucleic acid or protein in the basolateral medium by any known techniques in the art, such as PCR, RT-PCR, or ELISA.

In one embodiment, a HIV peptide that may be used for the evaluation of the virus transcytosis may be the p24 peptide that may be detected by means of an ELISA assay using, for example, a kit provided by PASTEUR-SANOFI (FRANCE).

In a particular embodiment, when added to about 10⁶ HIV-1 infected PBMCs at a concentration of about 0.5 ng/ml to about 5 ng/ml and incubated for about 1 hour at about 17° C. or at about 4° C. before inoculation of the infected cells to the apical chamber, the monoclonal antibody of the invention or a fragment thereof, may inhibit the virus transcytosis initiated by the addition of 10⁶ HIV-1⁺ PBMC to the apical chamber by at least about 50%, in particular by at least about 75% and more particularly by at least about 95% relative to the virus transcytosis performed without antibody.

In another embodiment, the antibody of the invention, or fragment thereof, may be added to the apical chamber prior to the addition of the HIV-infected PBMCs. In such embodiment, the inhibition of the transcytosis may be at least of about 50% relative to the virus transcytosis performed without antibody.

As an example, the Fab and Fd of clone 69, which are also objects of the instant invention, may inhibit the HIV transcytosis by respectively at least about 70% and about 80%.

In a particular embodiment, Fab and Fd of clone 69, which are also objects of the instant invention, are able to bind specifically to gp41 expressed at the surface of HIV-1 (JRCSF clone R5) infected PBMCs as measured by flow cytometry analysis, according to conventional methods known in the field.

In another embodiment, the monoclonal antibodies according to the invention or fragments thereof, may display the ability to inhibit the HIV-infection of CD4+ T cells.

In a particular embodiment, after incubation of the virus at about 1 ng/ml for about 30 min at about 37° C. with the antibody of the invention or fragment thereof, the infection of CD4+ T cells by HIV may be inhibited by at least about 75%, and more particularly by at least about 95% relative to the HIV-infection of CD4+ T cells performed without antibody or with a non specific antibody that does not recognize the virus.

As example, the Fab clone 69 which is an object of the instant invention may inhibit the HIV-infection of CD4+ T cells by at least 95%.

### Nucleic Acids, Vectors and Host Cells

The antibody of the invention has been identified by screening a Fab IgA phage display library obtained as described in the experimental section.

The combinatorial library production and manipulation methods have been extensively described in the literature and are from general knowledge of the skilled person in the art.

The combinatorial Fab phage display library has been screened in a two-steps approach.

In the first step, the Fab phage display library has been screened on peptide P1 having the sequence set forth as SEQ ID NO:12 immobilized on a solid support (such as enzyme-linked immunosorbent assay (ELISA) plates), in order to deplete the library of the antibodies recognizing this peptide.

This first step may be carried out with an antigen concentration of about 100 μM, at which the peptide P1 adopts a dimer/tetramer oligomerisation state.

In the second step, the Fab phage display library has been screened on a gp41 engineered loop protein by means of an iterative panning on antigen immobilized on a solid support, method known as micropanning, described in AZZAY & HIGHSMITH (Clinical Biochemistry, 2002, 35:425-445).

The gp41 engineered loop protein that may be used to screen the Fab phage display library of the invention may have, for example, the amino acid sequence set forth as SEQ ID NO:13, or a functional analogue thereof.

A starting amount may be for example from about 2 to about 1 μg then the amount may be divided twice at each panning round.

The detection of the interaction between the antigen, namely a gp41 engineered loop protein, and the antigen-binding domain displayed on the outside of the bacteriophages may be evaluated by any known techniques in the field. For example, an ELISA assay may be used wherein a gp41 engineered loop protein is coated in wells of a plate and then phages from the library are applied.

The genes encoding the antigen-binding site, which are unique to each phage, may then be recovered from the phage nucleic acid of the selected phage, sequenced and used to construct genes for a complete antibody molecule or analogue thereof, such as Fab fragment, F(ab′)₂, or scFv as above-described.

The sequence of the genes encoding the antigen-binding site recovered from the selected phage may be sequenced according to any known technique from the skilled person in the art.

For example, sequencing may be carried out by using an automated DNA sequencer with a Taq fluorescent dideoxynucleotide terminator cycle sequencing kit (Applied Biosystems). Double strand DNA may be prepared, for example, from bacteria and sequencing may be carried using a set of primers that anneal specifically to the vector used for cloning of the H and L chains of Fab, up and down stream of each Fab chain. For example, when using the pComb3X vector, the primers having the sequences set forth in the Table II may be used.

Therefore, according to another embodiment, the present invention also relates to nucleic acids molecules such as cDNA, RNA, and the like encoding the heavy and light chain variable region amino acid sequences of the invention where these sequences are set forth as, respectively, SEQ ID NO:7 and SEQ ID NO:8.

In particular, the nucleic acid sequences of the invention may be for the H chain variable region the sequence set forth as SEQ ID NO:9, and for the L chain variable region the sequence set forth as SEQ ID NO:10.

Of course, due to the degeneracy of the genetic code, variations may be contemplated in the nucleic acid sequence of the heavy and light chains variable regions respectively shown in SEQ ID NO:9 and SEQ ID NO:10 which will result in nucleic acids sequences that may be capable of directing production of antibodies or functional analogues thereof comprising the heavy chain variable region amino acid sequence shown in SEQ ID NO:7 and the light chain variable region amino acid sequence shown in SEQ ID NO:8.

According to an embodiment, the invention also relates to a nucleic acids sequence encoding an antibody of the invention or a fragment thereof. This nucleic acid may be RNA, cDNA and the like. The nucleic acids sequence of the invention may be fused to other nucleic acids sequences to construct chimeric proteins by any known molecular biological techniques in the art. For example, the nucleic acids sequence of the invention may be fused to nucleic acid encoding for His-Tag or HA-Tag, which may be used, thereafter, for example, for purifying the antibodies of the invention.

According to another embodiment, the present invention also relates to an expression vector comprising a nucleic acid sequence encoding a monoclonal antibody in accordance with the invention, or a fragment thereof.

Such an expression vector may be used for the expression of the monoclonal antibody according to the invention, or fragment thereof, in various types of cells. Such a vector may be a plasmid or a viral vector. Such a vector may be capable of autonomous replication in a host cell, or may be integrated into the genome of a host cell.

As example of vectors that may convene for achieving the invention, mention may be made of pComb3X or pASK88.

The instant invention also contemplates the expression of an antibody of the invention or fragment thereof by means of various host cells, such as bacteria, mammal cells (CHO, or HEK293), insect cells (such as Sf9 cells), or plant cells (tobacco, tomatoes).

After expression, an antibody of the invention may be isolated and purified by any known techniques in the field. For example, an antibody may be expressed with a His-Tag or a HA-Tag, and may be thereafter purified on a Nickel column or with a specific antibody as usually performed in the art.

Therefore, the present invention is also directed to a host cell transformed with a nucleic acid sequence encoding for a monoclonal antibody in accordance with the invention or a fragment thereof. The host cell may be obtained according to any known technique of transformation known in the art, such as electroporation, calcium phosphate techniques, lipofection, infection with, for example, a recombinant virus. The present invention is directed to a host cell transformed with a nucleic acid sequence encoding for a monoclonal antibody in accordance with the invention or a fragment thereof, such as electroporation in the case of bacteria, or lipofection in the case of mammal cells (CHO).

### Pharmaceutical Composition

According to one embodiment, the invention is related to a pharmaceutical composition comprising as active agent an effective amount of at least one agent selected among an antibody of the invention or a fragment thereof, in particular the clone 69, an H chain variable region of the invention, a recombinant anti-HIV antibody of the invention or a fragment thereof, a nucleic acid according to the invention, an expression vector according to the invention or a host cell according to the invention, and a suitable carrier.

According to another embodiment, the above-described active agent may be used for manufacturing a medicament intended to be used in the prophylaxis and/or the treatment of HIV-infection, and in particular of HIV-1 infection.

The term “effective amount” means the minimal amount necessary to observe the expected effect i.e. a neutralization of HIV and/or prevention of HIV infection. A therapeutically or prophylactic effective amount of an antibody according to the invention, or a fragment thereof, for individual patient may be determined as being the amount of antibody given to the individual to arrive at the therapeutic or prophylactic effect (i.e. reduction or prevention of the infection) while minimizing side effects. The effective amount may be measured by serological decreases in the amount of HIV antigens in the individual.

An exemplary, non limiting range for a therapeutically or prophylactically effective amount of an antibody of the invention, or a fragment thereof, is of about 0.1-100 mg/kg, more particularly about 0.5-50 mg/kg, more particularly about 1-20 mg/kg and even more particularly about 1-10 mg/kg.

According to another embodiment, the monoclonal antibody of the invention, or fragments thereof, or a pharmaceutical composition of the invention may be used in passive immunotherapy, by administering to an individual liable to be infected or susceptible to be exposed to HIV, or having been exposed to HIV a therapeutically effective amount of at least an antibody according to the invention or a fragment thereof, or a pharmaceutical composition of the invention. The passive immunotherapy of the invention may be practiced on individuals exhibiting AIDS or related conditions caused by HIV infections or individuals at risk of HIV infections.

According to one embodiment, the passive immunotherapy of the invention may also be practiced prophylactically, i.e. before a susceptible exposure to HIV.

The pharmaceutical composition of the invention may be administered per os, parenterally (intra-veinously or intra-nasaly or the like), topically, rectally vaginally, or the like.

Therefore, the pharmaceutical composition of the invention may be prepared under various galenic forms, such as injectable or infusible sterile solution, dispersion or suspension, tablet, pills, powders, liposomes, suppositories and cream.

The suitable carrier to be used in the manufacture of the pharmaceutical composition of the invention is to be adapted according to the galenic form intended to be carried out. Such suitable carriers include, but are not limited to, water, saline, phosphate buffered saline, dextrose, glycerol and the like, as well as combinations thereof. Pharmaceutically acceptable substances such as wetting or emulsifying agents, preservatives or buffers may also be included in the pharmaceutical compositions of the invention.

The pharmaceutical composition of the invention may also include, in addition of the active agents of the invention, other active agents against HIV, such as antiretroviral drugs. According to another embodiment, such additional antiretroviral drugs may be administered in a combination with the pharmaceutical composition of the invention, simultaneously, separately, or sequentially in time.

Within another embodiment, the antibody of the invention, or fragment thereof, may also be labelled for therapeutic purpose, in which case the labelling agent may be a drug conjugate or a toxin, such as a radioisotope or a radionuclide (¹³¹I, ⁹⁹Tc, ¹¹¹In or the like), pertussis toxin, taxol, cytochalasin B, doxorubicin and the like.

### Diagnostic Composition and Application

The monoclonal antibody of the invention or fragment thereof, may also be used as a diagnostic agent in a method for detecting in vitro a HIV strain, and in particular a HIV-1 strain, in a sample.

In one of its embodiment, the method of the invention comprises at least the step of:
a) contacting the sample with at least an antibody of the invention or a fragment thereof, under conditions suitable to form a complex between said antibody, or fragment thereof, and a gp41 protein or a functional analogue thereof, and
b) detecting the presence of said complex.

As example of gp41 protein that may be complexed with an antibody according to the invention, mention may be made of wild-type gp41 proteins such as for example the gp41 protein having the amino acid sequence set forth as SEQ ID NO:11, or functional analogue thereof, or modified gp41 proteins such as gp41 engineered loop protein, having for example the amino acid sequence set forth as SEQ ID NO:13, or functional analogues thereof.

The detection of the complex may be carried out by any immunoassay known in the art.

Such assays include, but are not limited to, radioimmunoassay, Western blot assay, immunofluorescent assay, enzyme immunoassay (such as ELISA), chemiluminescent assay, immunohistochemical assay and the like.

Additionally for the ease of detection, the monoclonal antibody of the invention, or fragment thereof, may be labelled by means of a detectable marker. As example of labelling agent, mention may be made of fluorescent compounds, such as fluorescein or rhodamine; enzymes such as horseradish peroxydase, beta-galactosidase or luciferase; biotin allowing detection through indirect measurement of avidin or streptavidin binding; or radiolabelled amino acid comprising, for example, as radioisotope or radionuclide ³H, ¹⁴C or ¹²⁵I.

According to another of its embodiments, the instant invention is also directed to a diagnostic composition comprising an antibody in accordance with the invention or a fragment thereof.

In order that this invention may be better understood, the following examples are set forth.

These examples are for purposes of illustration only and are not to be construed as limiting the scope of the invention in any manner.

### BRIEF DESCRIPTION OF THE DRAWINGS

FIG. 1: It represents immunoblot of gp41-derived HIV-1 protein exposed to anti-HIV-1 IgA-content cervico-vaginal secretion of Highly Exposed Persistently IgG Seronegative (HEPS) individuals. As a positive control, immunoblot of gp41 peptide was performed with serum obtained from HIV seropositive individuals. And as negative control, the same experiment was performed with serum of HIV seronegative individuals.

FIG. 2: It represents a schematic view of the plasmid pComb3X wherein the variable and constant regions of the heavy and light chains from the Fab IgA phage library have been inserted, respectively, between restriction enzyme sites Sac 1 and Xba 1, and between Xho 1 and Spe 1.

FIG. 3: It represents the inhibition of HIV-1 transcytosis across the endometrial HEC-1 cell line by selected IgA Fab clones. Negative control was obtained by performing the experiment without antibody (standard) or with a product resulting from the screening of a phage library obtained with the empty plasmid TOP1O. Positive control was achieved by using the 2F5 antibody IgA.

FIG. 4: It represents a dot blot performed with the clone Fab 69 and the antibody alpha IgA 2F5 on a wild-type gp41 protein of HIV strain HxB2 (amino acids from 546 to 682), a leucine zipper, BSA and lysozyme. After washing the nitrocellulose membranes, the antibodies proteins complexes were incubated with a mouse anti-His antibody for the Fab clone 69 and a mouse anti-human antibody for the 2F5 antibody. Both were revealed with a goat anti-mouse antibody coupled to a horseradish peroxydase followed by ECL and autoradiography.

FIG. 5: It represents the amino acid sequences of corresponding to the complementary determining sequences of a H chain variable region of an antibody according to the invention (CDR1 as SEQ ID NO:1, CDR2 as SEQ ID NO:2, and CDR3 as SEQ ID NO:3), and the CDRs of a L chain variable region of an antibody according to the invention, CDR1, CDR2 and CDR3 set forth as SEQ ID NO:4, SEQ ID NO:5, and SEQ ID NO:6.

FIGS. 6A and 6B: It represents the amino acid sequences and the nucleic acid sequences of the heavy chain variable region (SEQ ID NO:7 and SEQ ID NO:9) and of the light chain variable region (SEQ ID NO:8 and SEQ ID NO:10) of the clone Fab 69.

FIG. 7: It represents the amino acid sequences of the frame work region (FR) and complementary determining region (CDR) of the light and heavy chains of the Fab IgA clone 69 obtained using the IMTG program available free on line (and created by M. P. LEFRANC, France). IMGT/V-QUEST software (http://imgt.cines.fr).

FIG. 8: It represents the amino acid sequences corresponding to the wild-type gp41 protein, SEQ ID NO:11 (HIV-1 strain HxB2), the peptide P1 SEQ ID NO:12.

FIG. 9: It represents the amino acid sequences of the gp41 engineered loop protein SEQ ID NO:13.

Table I: It represents the sequences of the primers used to amplify the DNA obtained from the HEPS individuals.

Table II: It represents the sequences of the primers used to identify and sequence the nucleic acids sequences of Fab IgA from the phage display library.

### EXAMPLE 1

### Identification of Anti-HIV-1 IgA in Cervico-Vaginal Secretions of Highly Exposed Persistently IgG Seronegative (HEPS) Individuals

Cervical secretions from 56 HEPS originated from Cambodia were tested for HIV-envelope glyco-protein S-IgA. Secretions were collected after two days of sexual abstinence using 3 ml of sterile PBS. Samples were centrifuged and frozen and stored at −80° C. Sample contamination by sperm was measured using the seminal fluid detection assay (SEMA; HUMANGEN FERTILITY DIAGNOSTICS, Charlottesville, Va.) according to the manufacturer but optimized by increasing the incubation times and using O-phenylenediamine dihydrochloride as substrate. Contaminated samples were discarded.

The presence of specific IgA antibodies to HIV was tested by western blot using a commercially available kit (New Lav Blot1, Sanofi-Pasteur, France) using procedure according to the manufacturer.

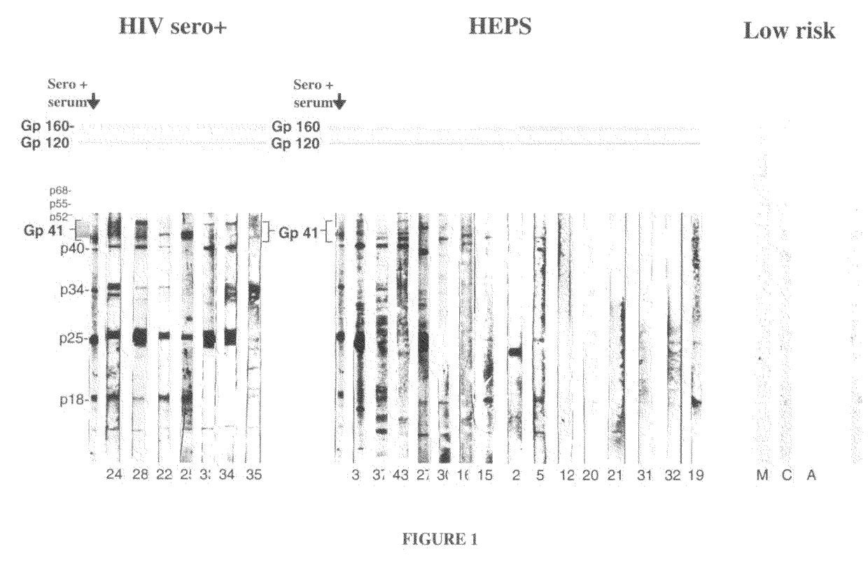

Among the tested samples, 22 contained a high level of anti-HIV envelope gp41 Secretory IgA (S-IgA) (FIG. 1).

### EXAMPLE 2

### Construction of a Combinatorial Phage Display Library Expressing Fab of IgA

Mucosal B cells of the 22 identified HEPS of the example 1 were used to construct a combinatorial phage display library expressing Fab of IgA.

Total RNA was prepared from an enriched B-cells population form cervico-vaginal secretions of the 22 HEPS individuals using standard techniques. Total RNA (60 μg) was purified from cervical B cells by a rapid single step guanidinium isothiocyanate/phenol chloroform-based RNA isolation (CHOMCZYNSKI & SACCHI, Anal. Biochem., 1987, 162:156-9.). This total RNA was transformed in complementary DNA (cDNA) using reverse transcriptase-polymerase chain reaction (RT-PCR). The obtained cDNA library was amplified using mucosal Fab IgA specific primers set forth in table I.

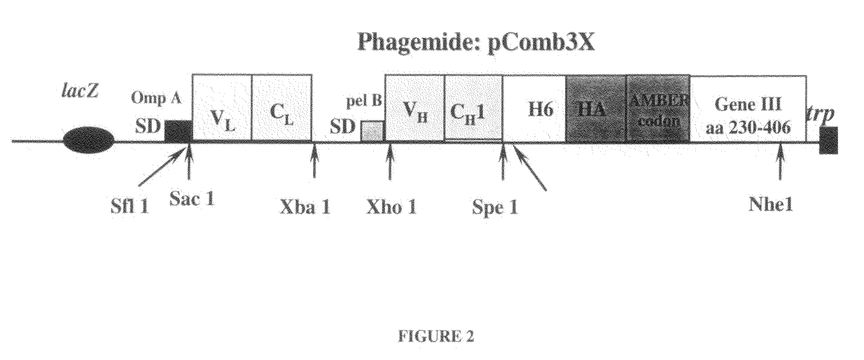

The amplified DNA was then digested with Sac I and Xba 1 for light chain (L) and Xho 1 and Spe 1 for heavy chain (H), and the digested products were inserted in the vector pComb3X (BARBAS et al., Proc Natl Acad Sci USA, 1991, 88:7978-82) digested with the same enzymes. The digested vector and the digested PCR product were ligated using DNA ligase of phage T4 (4 inserts plus 1 vector plus T4DNA ligase, overnight at 14° C.) (FIG. 2). The obtained recombinant plasmids were then used to transform bacteria E. coli (pilus+, male) TG1 by means of electroporation.

The library of antibody cDNAs in the culture after transformation of the bacteria was then expressed on bacteriophage by super infection with the helper phage VCS-M13 (10¹² pfu) (Stratagene, La Jolla, Calif.)

The phage preparations were harvested. Phages were precipitated by addition of 20% (wt/vol) polyethylene glycol 8000 and 2.5 M NaCl followed by incubation on ice for one hour. After centrifugation phage pellet was resuspended in phosphate-buffered saline (PBS) and microcentrifuged for few minutes to pellet debris.

Restriction digest analysis of colonies from the titer plates indicated that approximately 35% of the phages contain Fab IgA and that the library contained titers were typically close to 10⁷ cfu/ml, and 10⁷ different clones.

### EXAMPLE 3

### Screening of the Fab IgA Phage Library on GP41 Engineered Loop Protein (SEQ ID NO:13)

The Fab IgA phage display library has been screened by means of micro panning, in which the antigen concentration was the varying condition at each round. The antigen used for the screening was a gp41 engineered loop protein (SEQ ID NO:13).

Prior to screening of the Fab IgA phage display library with the gp41 engineered loop protein (SEQ ID NO:13), a first panning round was performed with the peptide P1 (SEQ ID NO:12) (ALFSEN & BOMSEL, 2002, J. Biol. Chem., 277:25649-59; obtained from Eurogentec, Belgium) at an amount of 20 μg (100 μM) to which this peptide is in oligomeric state. Such protocol allows to discard any Fab IgA liable to bind to the peptide P1 in an oligomeric state.

The starting amount of gp41 engineered loop protein used was 1 μg, and the amount was divided by 2 at each round to reach finally the amount of 125 ng, at the fourth.

Wells of a 96-plates (Exiqon peptide Immobilirez, 10202-111-10) were coated with 1 μg of gp41 engineered loop protein, then blocked with BSA for 2 hours at 37° C. The concentrated phages prepared from the library were then applied to the wells at 10¹²-10¹³ pfu, for 2 hours at 37° C. Unbound phage were then removed by vigorous washing, for the first and second rounds of selection, 10 times with PBS containing 0.1% Tween-20, then 10 times with PBS to remove the detergent. For the subsequent rounds of selection washing was carried out 20 times with PBS containing 0.1% Tween-20, then 20 times with PBS. The specific elution of phages bearing epitopes of gp41 engineered loop protein-binding surface Fabs was performed by treatment with 0.1 M glycine-HCL, adjusted to pH 2.2 for 10 min. The elution was immediately neutralized and used to infect 2 ml of fresh E. coli TG1 bacterial culture (OD₆₀₀=1). Bacteria were incubated at 30° C. for 30 minutes, culture volume was increased and culture was incubated in at 37° C. shaker for 1 hr. and thereafter 10¹² pfu/ml of VCS-M13 helper phage was added for overnight production of recombinant phages. The eluted phages were amplified between each panning round.

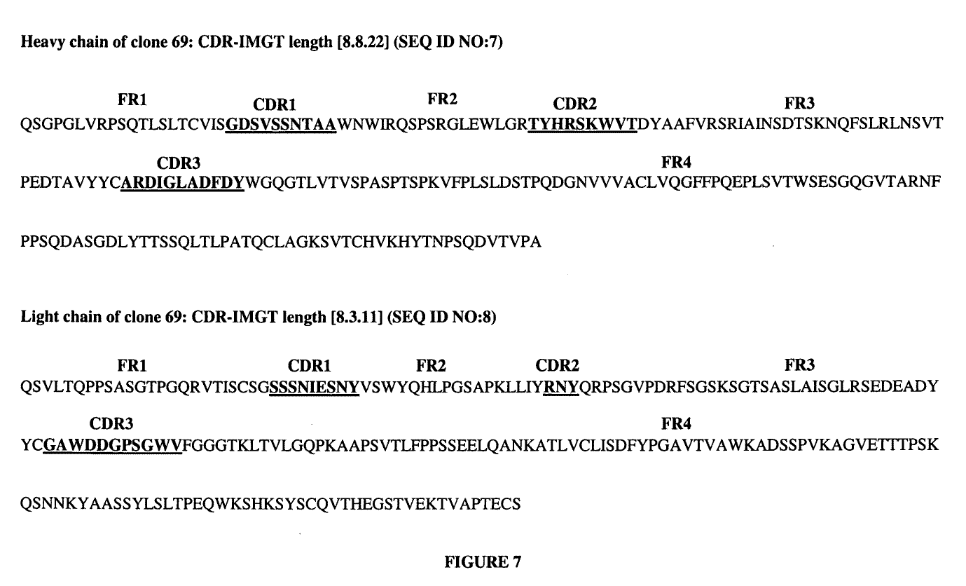

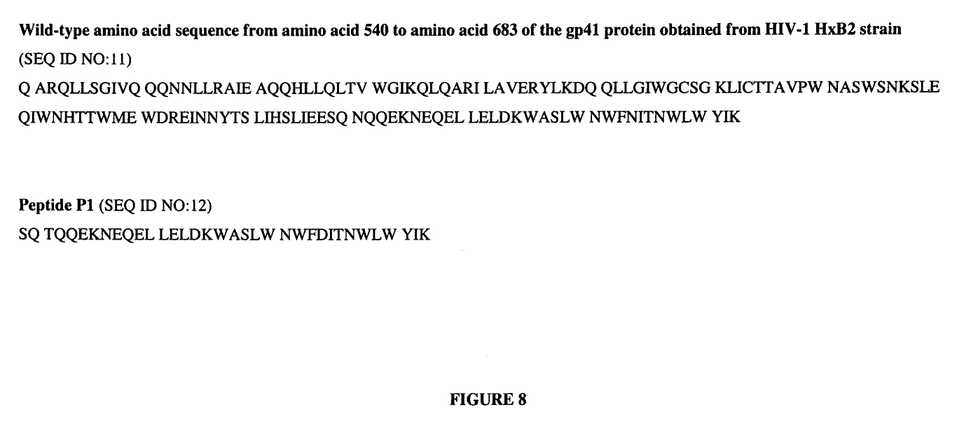

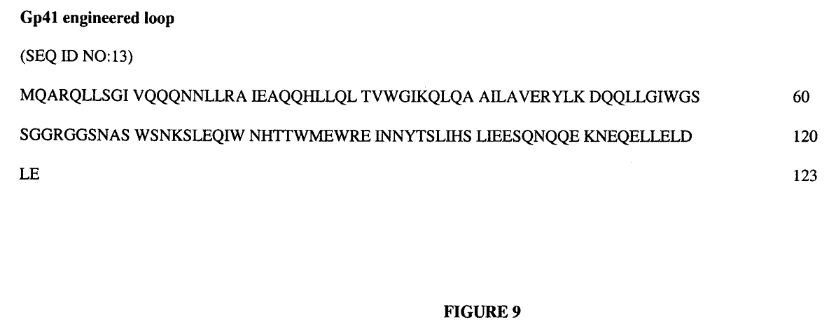

After panning, individual clones from the four rounds were grown and the presence of Fab was monitored by PCR using specific primers set forth in Table II which hybridize to the vector upstream and downstream to the Fab light and heavy chains. Clone containing Fab IgA were converted to soluble Fab by transforming in an E. coli amber non-suppressor strain and sequenced to determine the variable region sequences of Heavy (H) and Light (L) chains using IMGT/V-QUEST software (FIG. 7).

### Sequencing of Immunoglobuline Genes

Sequencing was done using an automated DNA sequencer with a Taq fluorescent dideoxynucleotide terminator cycle sequencing kit (Applied Biosystems). Double strand DNA was prepared from bacteria and sequencing was carried out using a set of primers (see Table II) that anneals specifically to the pComb3X vector up and down stream of each Fab chain.

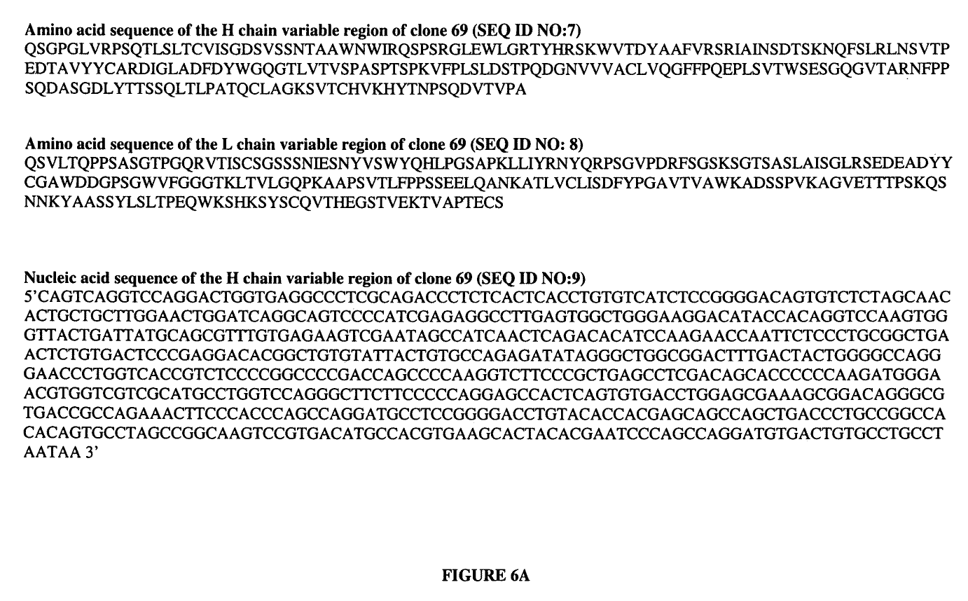

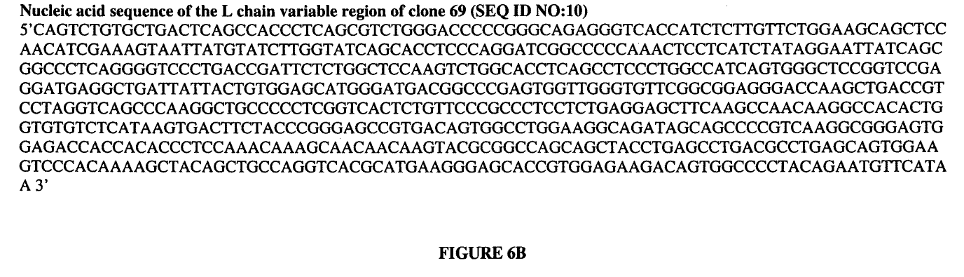

FIGS. 6A and 6B show the result of the sequencing of the H and L chain variable regions of clone 69 (SEQ ID NO:9 and SEQ ID NO:10).

FIG. 7 shows the identification in the corresponding amino acid sequences of the complementarity determining regions using the IMGT software.

The vector used for cloning has an amber codon between the tag (His-tag and HA-tag) sequences and the phage protein pIII providing the opportunity to either produce the antibody fused with the coat protein of the phage, using a E. coli suppressor strain such as TG1, or to produce soluble antibodies by expressing the vector in a non-suppressor strain, such as TOP-10. Taking into account this advantage, DNA of phages was used to transform TOP-10 E. coli (amber non-supressor-strain) in order to express soluble Fab fragments without the pIII fusion protein. Fab expression was induced using 1 mM Isopropyl β-D-thiogalactopyranoside (IPTG)

For large-scale expression of the monoclonal antibody of the invention the whole operon was transferred from the pComb3X to the vector pASK88 (SKERRA A., Gene, 1994, 151(1-2): p. 131-5; SKERRA A., Gene, 1994, 141(1): p. 79-84; SKERRA A., et al., Biotechnology (N Y), 1991. 9(3), p. 273-8; SKERRA A. & A. PLUCKTHUN, Protein Eng, 1991, 4(8): p. 971-9).

Fab DNA of the monoclonal antibody of the invention was amplified with specific primers introducing restriction sites needed for subcloning into the pASK88 vector. The amplification products were purified by agarose gel electrophoresis and cut with the restriction enzymes PstI and NcoI in the case of heavy chain and SacI and HindIII in the case of light chain. The Fd (VH-CH1) and light chain (VL-CL) genes were separately inserted and ligated into pASK88 digested with the same restriction enzymes using standard protocols. The ligations were transformed into thermocompetent JM83 (provided by Dr. Skerra) bacterial cells and plated for separation in individual clones. Plasmid DNA for several clones was analyzed by restriction digest and, for some of them, by double strand sequencing using specific primers (SKERRA, A., Gene, 1994, 151(1-2): p. 131-5; SKERRA, A., Gene, 1994, 141(1): p. 79-84; SKERRA, A., et al., Biotechnology (N Y), 1991. 9(3), p. 273-8; SKERRA, A. & A. PLUCKTHUN, Protein Eng, 1991, 4(8): p. 971-9).

The expression vector pASK88 was designed for the convenient cloning of immunoglobulin variable domain genes as well as periplasmic secretion of the corresponding Fabs fragment in Escherichia coli. On this plasmid, expression was under control of the tetracyclin promoter.

Large quantities of the monoclonal antibody of the invention were obtained using this vector. Preparative expression was performed on the 1-litre scale, employing E. coli K-12 JM83 as expression host. The cells were grown to a mid-log phase, and the Fab expression was then induced by 0.2 mg/L anhydrotetracycline for 4 h or overnight.

Purification of the monoclonal antibody of the invention was performed on Ni-NTA Spin Columns (Qiagen) by interaction with the His-tag. The periplasmic fraction filter sterilized was applied on the column and a gradient of 250-500 mM imidazole in chromatography buffer was applied to collect the samples.

### EXAMPLE 4

### ELISA Assay Performed with Soluble IgA Fab Clones from the Phage Display Library

Elisa was performed with soluble IgA Fab obtained from TOP-10F′ bacteria (pComb3x system) and with soluble IgA Fab obtained from K-12 JM83 bacteria (pASK88 system).

Wells of plates (Exiqon peptide Immobilirez, 10202-111-10 or NUNC, 439454) were coated with 100 ng of gp41 engineered loop protein (SEQ ID NO:13) and blocked with BSA for 2 hours at 37° C. Then, the purified IgA at 2 ng/ml were added and incubated for 2 hours at 37° C. Detection was done by mouse anti-Ha antibody (clone 12CA5, Roche) or anti-His (PentaHis, Qiagen 34660) followed by horseradish peroxydase goat anti-mouse antibody (Caltag Laboratories, H1003). The enzymatic reaction was developed by adding TMB (3,3′,5,5′-tetramethyl-benzidine, Kikergaard & Perry Laboratories Inc.) as a substrate and absorbance was measured at 450 nm after addition of 1M acid phosphoric using an ELISA reader. The positive control was 2F5 antibody and the negative control was a clone obtained after panning that does not recognize gp41 engineered loop protein by ELISA.

### EXAMPLE 5

### Inhibition of Transcytosis

HIV-1 transcytosis across epithelial cells and neutralisation of transcytosis by antibodies were performed on intestinal cell line HEC-1 grown as a tight, polarized monolayer for 7 days on a permeable filter support (0.45 μm pore size) forming the interface between two independent chambers, the upper one bathing the apical (luminal) surface of the epithelial monolayer and the lower one bathing the basolateral (serosal) surface.

PBMC were obtained and prepared as described in LAGAYE et al., (J. Virol, 2001, 75:4780). Then PBMCs were activated with phytohemagglutinin (PhA) for 48 h and inoculated with HIV-1 JRCSF clone R5 or YU2 and used at day 7 post infection. Purified S-IgA (5 ng/ml) were added to the apical chamber and incubated for 10 min at 37° C.

To initiate virus transcytosis, 2.10⁶HIV-1⁺ PBMC were added to the apical chamber. Contact between HIV-1⁺ PBMC and the epithelial cell monolayer resulted in rapid budding of the HIV-1 virions, followed by their transcytosis from the apical to the basolateral pole of the epithelial cells. After 2 hours, inhibition of transcytosis by antibody was determined by detection of the protein p24 of HIV in the basolateral medium by ELISA (Coulter, France or PASTEUR SANOFI, FRANCE). The level of p24 in the absence of antibody or in the presence of a gp41 engineered loop protein non specific Fab or in the presence of the control IgA 2F5 was measured as negative and positive control respectively with a value of 100, 98, and 35% respectively. The value of the negative control was taken as 100% of transcytosis and used to express the results.

The experiments were performed in three independent experiments.

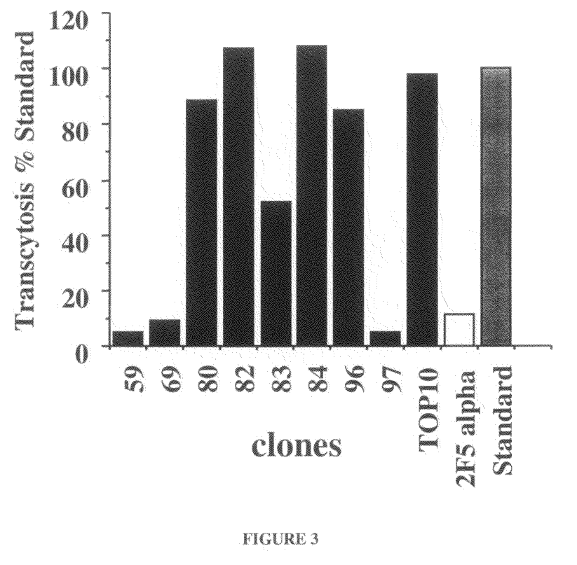

The results allow identifying 3 clones that were able to inhibit the HIV transcytosis by more than 50% among which was the clone 69 (FIG. 3).

### EXAMPLE 6

### Dot Blot

The BSA, the leucine zipper, the lysozyme and the wild-type gp41 protein of the HIV strain HxB2 (amino acids from 546 to 682, obtained from ABI) were doted to nitrocellulose membranes, respectively, at 100 ng/ml each. Thereafter, the Fab clone 69, at 5 ng/ml, or the antibody 2F5 alpha IgA, at 2 μg/ml 1:2500, were incubated with the nitrocellulose membranes.

After washing the nitrocellulose membranes (Western Blocking reagent, Roche: 1% for Blocking, and washing, 0.1% for antibody binding, according to manufacturer instruction), the immune complexes were incubated with a mouse anti-His antibody (Qiagen, 0.2 mg/ml, 1:5000) for the Fab clone 69 and with a mouse anti-human antibody for the 2F5 alpha IgA. Both were revealed a goat anti-mouse antibody coupled to a horseradish peroxydase (Caltag Laboratories, H1003 1/4000) followed by ECL and autoradiography (ECL, Amersham, according to manufacturer instructions).

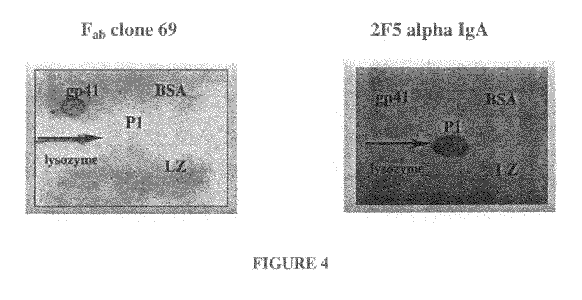

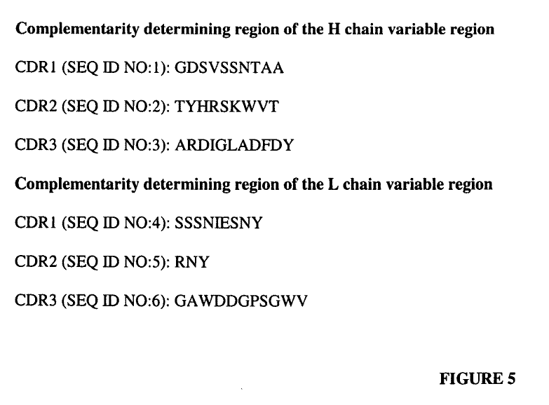

FIG. 4 is a representative result of an experiment repeated twice.

TABLE I

Primers used for the amplification of human α,
(Fd) and K and λ sequences
α1
V_{H}5′ primers (5′-3′)

| | | |
|---|---|---|
| V_{H1a} | CAG GTG CAG CTC GAG CAG TCT | (SEQ ID NO: 14) |
| | GGG | |
| V_{H1f} | CAG GTG CAG CTG CTC GAG TCT | (SEQ ID NO: 15) |
| | GGG | |
| V_{H2} | CAG GTC ACC TTG CTC GAG TCT | (SEQ ID NO: 16) |
| | GGT | |
| V_{H3a} | GAG GTG CAG CTC GAG GAG TCT | (SEQ ID NO: 17) |
| | GGG | |
| V_{H3f} | GAG GTG CAG CTG CTC GAG TCT | (SEQ ID NO: 18) |
| | GGG | |
| V_{H4f} | CAG GTG CAG CTG CTC GAG TCG | (SEQ ID NO: 19) |
| | GG | |
| V_{H4g} | CAG GTG CAG CTA CTC GAG TGG | (SEQ ID NO: 20) |
| | GG | |
| V_{H5} | GAG GTG CAG CTC GAG CAG TCT | (SEQ ID NO: 21) |
| | GG | |
| V_{H6} | CAG GTA CAG CTC GAG CAG TCA | (SEQ ID NO: 22) |
| | GG | |
| V_{H7} | CAG GTG CAG CTC GAG CAA TCT | (SEQ ID NO: 23) |
| | GG | |

C_{H}3′ primers (5′-3′)

| | | |
|---|---|---|
| CHA-1 | AGT TGA ACT AGT TGG GCA GGG | (SEQ ID NO: 24) |
| | CAC AGT CAC | |
| CHA-2 | AGT TGA ACT AGT TCG GCA GGG | (SEQ ID NO: 25) |
| | AAC AGT CAC | |

V_{K}5′ primers

| | | |
|---|---|---|
| V_{K1a} | GAC ATC GAG CTC ACC CAG TCT | (SEQ ID NO: 26) |
| | CCA | |
| V_{K1s} | GAC ATC GAG CTC ACC CAG TCT | (SEQ ID NO: 27) |
| | CC | |
| V_{K2a} | GAT ATT GAG CTC ACT CAG TCT | (SEQ ID NO: 28) |
| | CCA | |
| V_{K2b} | GAT ATT ACC CAG ACT CCA | (SEQ ID NO: 29) |
| V_{K3a} | GAA ATT GAG CTC ACG CAG TCT | (SEQ ID NO: 30) |
| | CCA | |
| V_{K3b} | GAA ATT GAG CTC AC(G/A) CAG | (SEQ ID NO: 31) |
| | TCT CCA | |
| V_{K4} | GAC ATC ACC CAG TCT CCA | (SEQ ID NO: 32) |
| V_{K5} | GAA ACG GAG CTC ACG CAG TCT | (SEQ ID NO: 33) |
| | CCA | |
| V_{K6} | GAA ATT GAG CTC ACT CAG TCT | (SEQ ID NO: 34) |
| | CCA | |

V_{λ}5′ primers

| | | |
|---|---|---|
| V_{L1} | CAG TCT GAG CTC ACG CAG | (SEQ ID NO: 35) |
| | CC(G/A) CCC TC | |
| V_{L2} | CAG TCT GAG CTC ACT CAG CCT | (SEQ ID NO: 36) |
| | GCC TC | |
| V_{L3} | GCC TCC TAT GAG CTC ACT CAG | (SEQ ID NO: 37) |
| | CCA | |
| V_{L4a} | CAG CCT GAG CTC ACT CAA TCA | (SEQ ID NO: 38) |
| | TCC TC | |
| V_{L4b} | CAG CCT GAG CTC ACT CAG CCC | (SEQ ID NO: 39) |
| | CCG TC | |
| V_{L5} | CAG CCT GAG CTC ACT CAG CCG | (SEQ ID NO: 40) |
| | (G/T)CT TCC | |
| V_{L6} | AAT TTT GAG CTC ACT CAG CCC | (SEQ ID NO: 41) |
| | CAC | |
| V_{L7} | CAG ACT GAG CTC ACT CAG GAG | (SEQ ID NO: 42) |
| | CCC | |
| V_{L8} | CAG AGT ACC CAG GAG CCA TCG | (SEQ ID NO: 43) |
| | TTC | |
| V_{L9} | CAG CCT GAG CTC ACT CAG CCA | (SEQ ID NO: 44) |
| | CCT TC | |
| V_{L10} | CAG GCA GAG CTC ACT CAG CCA | (SEQ ID NO: 45) |
| | CCC TCG | |

CL3′ primers
κ3′ primers

| | | |
|---|---|---|
| CLK | TCC TTC TAG ATT ACT AAC ACT | (SEQ ID NO: 46) |
| | CTC CCC TGT TGA AGC | |

λ3′ primers

| | | |
|---|---|---|
| CL2 | CGC CGT CTA GAA TTA TGA ACA | (SEQ ID NO: 47) |
| | TTC TGT AGG | |

TABLE II

Primers used to identify and sequence the
nucleic acid sequences of Fab IgA from the
phage display library

| | |
|---|---|
| OmpA | AAG ACA GCT ATC GCG ATT GCA G |
| | (SEQ ID NO: 48) |
| PelSeq | ACC TAT TGC CTA CGG CAG CCG |
| | (SEQ ID NO: 49) |
| PelSeq Rev | GAG CAG CTG CAC CTC GGC CAT G |
| | (SEQ ID NO: 50) |
| Rev | GCC CCC TTA TTA GCG TTT GCC ATC |
| | (SEQ ID NO: 51) |

1. An isolated monoclonal antibody or a fragment thereof comprising in the H chain variable region at least one complementarity determining region (CDR) selected from the group consisting of CDR1, CDR2 and CDR3 having, respectively, an amino acid sequence set forth as SEQ ID NO:1, SEQ ID NO:2, SEQ ID NO:3 or functional analogues thereof.

2. The monoclonal antibody or a fragment thereof according to claim 1, wherein it comprises a L-chain variable region comprising at least one CDR selected from the group consisting of CDR1, CDR2 and CDR3 having, respectively, an amino acid sequence set forth as SEQ ID NO:4, SEQ ID NO:5, SEQ ID NO:6 or functional analogues thereof.

3. The monoclonal antibody according to claim 1, wherein it does not recognize a peptide of the amino acid sequence set forth as SEQ ID NO:12.

4. An isolated monoclonal antibody or a fragment thereof, recognizing a gp41 engineered loop protein having the amino acid sequence set forth as SEQ ID NO:13 and not recognizing a peptide of the amino acid sequence set forth as SEQ ID NO:12.

5. The monoclonal antibody or a fragment thereof, according to claim 1, wherein it is an IgA.

6. The monoclonal antibody or a fragment thereof, according to claim 1, wherein it is a human antibody.

7. The monoclonal antibody or a fragment thereof, according to claim 1, wherein the heavy chain variable region has the amino acid sequence set forth as SEQ ID NO:7 and the light chain variable region has the amino acid sequence set forth as SEQ ID NO:8.

8. The monoclonal antibody or a fragment thereof, according to claim 1, wherein it has the ability to neutralize HIV.

9. The monoclonal antibody or a fragment thereof, according to claim 8, wherein the neutralized HIV is a HIV-1 strain.

10. A H chain variable region, wherein it comprises at least one CDR selected among CDR1, CDR2 and CDR3 having, respectively, an amino acid sequence set forth as SEQ ID NO:3, SEQ ID NO:4, SEQ ID NO:5 or functional analogues thereof.

11. The H chain variable region according to claim 10, wherein it does not recognize a peptide of an amino acid sequence set forth as SEQ ID NO:12.

12. The H chain variable region according to claim 10, wherein it recognizes a gp41 engineered loop protein having an amino acid sequence set forth as SEQ ID NO:13 and not recognizing a peptide of the amino acid sequence set forth as SEQ ID NO:12.

13. The H chain variable region according to claim 10, wherein it is comprised in a recombinant anti-HIV antibody or a fragment thereof.

14. An isolated nucleic acid sequence encoding a monoclonal antibody or a fragment thereof as defined according to claim 1.

15. The nucleic acid sequence according to claim 14, wherein the nucleic acid sequence of the heavy chain is set forth as SEQ ID NO:9.

16. The nucleic acid sequence according to claim 14, wherein the nucleic sequence of the light chain variable region is set forth as SEQ ID NO:10.

17. An expression vector comprising a nucleic acid sequence as defined according to claim 14.

18. A host cell transformed with a nucleic acid sequence as defined according to claim 16.

19. A pharmaceutical composition, comprising:
a suitable carrier; and
an active agent, the active agent comprising an effective amount of an agent selected from the group consisting of:
a) an isolated monoclonal antibody or a fragment thereof comprising in the H chain variable region at least one complementarity determining region (CDR) selected from the group consisting of CDR1, CDR2 and CDR3 having, respectively, an amino acid sequence set forth as SEQ ID NO:1, SEQ ID NO:2, SEQ ID NO:3 or functional analogues thereof;
b) an isolated nucleic acid sequence encoding a monoclonal antibody or a fragment thereof as defined in (a);
c) an expression vector comprising an isolated nucleic acid sequence encoding a monoclonal antibody or a fragment thereof as defined in (a); and
d) a host cell transformed with an isolated nucleic acid sequence encoding a monoclonal antibody or a fragment thereof as defined in (a).

20-22. (canceled)

23. A method for providing passive immunotherapy to an individual liable to be infected with HIV comprising administering to said individual a therapeutically effective amount of at least an antibody or a fragment thereof, as defined according to claim 1.
